(19) United States
(12) Patent Application Publication
(10) Pub. No.: US 2003/0082605 A1
(43) Pub. Date: May 1, 2003

(54) Genomic DNA detection method and system thereof

(76) Inventor: Timothy A. Hodge, Cordova, TN (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 10/233,759

(22) Filed: Sep. 3, 2002

Related U.S. Application Data
(63) Continuation-in-part of application No. 09/945,952, filed on Sep. 4, 2001.
(60) Provisional application No. 60/230,371, filed on Sep. 6, 2000.

(51) Int. Cl. C12Q 1/68; C12M 1/34; C12M 3/00

(52) U.S. Cl. 435/6; 435/287.2

(57) ABSTRACT

A method to directly detect eukaryotic or prokaryotic genomic DNA is disclosed. The invention relates to a method for printing, immobilizing, hybridizing and directly detecting a target nucleic acid sequence in a sample of methylated genomic DNA. Additionally, this invention provides a system for the detection of a target nucleic acid sequence including a flat substrate to bind methylated DNA in discrete patterns and a plurality of labeled target binding probes specific for a target nucleic acid sequence.

### CROSS-REFERENCE TO RELATED APPLICATIONS

This application is a continuation-in-part of U.S. patent application Ser. No. 09/945,952 filed Sep. 4, 2001, which is a continuation-in-part of U.S. Provisional Application Serial No. 60/230,371 filed Sep. 6, 2000. The entire disclosure of which is hereby incorporated by reference.

### BACKGROUND OF THE INVENTION

1. Field of the Invention

This invention relates to a system for applying, immobilizing, hybridizing and detecting a target nucleic acid sequence within a sample of genomic DNA that has been applied to a microarray substrate.

2. Description of the Related Art

The genome of a eukaryote is composed of a double-stranded DNA. The genome contains both the exon coding regions of the DNA as well as the noncoding intron regions. The number of base pairs for the mammalian genome far exceeds the hundred of base pairs for PCR and EST or several thousand for cDNA's. It is not uncommon to find mammals such as primates and mice with 3×10⁹ base pairs. Additionally, the eukaryote genome is methylated whereas the PCR amplicons, EST and cDNAs are not.

Polymerase Chain Reaction (PCR) is a commonly used assay that challenges the entire genome for a target(s) of interest. This methodology is enzyme dependent that utilizes the genomic DNA as a template to which specific primer pairs hybridize. The Thermus aquaticus (Taq) enzyme has polymerase activity which specifically adds dNTP's to the end of the primer forming two double stranded pieces of DNA. The heating conditions are such that the two new double stranded amplicons are separated and serve as new templates. The reaction continues to amplify the DNA is an exponential fashion. These amplicons are generally several hundred base pairs long. The amplicons are typically separated by electrophoresis in a gel, stained and visualized with ultraviolet light.

Historically, it has been possible to print polymerase chain reaction (PCR) amplicons, cDNAs and expression sequence tags (EST) onto a substrate. The forementioned genetic subsets only represent a small portion of the entire genome. As an example, PCR amplicons and EST are generally only hundreds of base pairs long. Additionally, cDNA are genetic elements which also only represent a portion of the genome which codes for proteins. cDNA do not contain introns but only exons.

PCR is currently a widely used technology to specifically detect genetic sequences of interest. PCR methodology is a process where enzymes manufacture multiple copies of the genetic sequence. The high number of copies allows one skilled in the art to separate these fragments and stain them so they can be visualized. PCR makes copies of original genome elements of interest so it is an indirect way to detect genetic sequences.

PCR reactions are susceptible to failure for a variety of reasons. Failure can be attributed to the PCR oligonucleotide primer becoming nonfunctional or demonstrates an inability to bind to the target. Conversely, nonspecific hybridization can occur producing a final fragment in electrophoresis that appears to be the correct size but indeed is the incorrect sequence. The PCR reaction is enzyme dependent therefore any degradation to the enzyme will inhibit the reaction. Additionally, the salt stringency in the environment must be optimized or failure can occur. PCR reactions will fail if the proper heating environment is not achieved during annealing, separation and extension phases of the reaction.

### BRIEF SUMMARY OF THE INVENTION

The present invention provides a unique solution to the above described problems by providing a method and system for applying, immobilizing, hybridizing and detecting methylated DNA on a flat microarray substrate. More specifically, present invention provides a method for detecting a target nucleic acid sequence involving the steps of applying a plurality of samples of methylated DNA on to a flat substrate to form discrete patterns of the methylated DNA on the flat substrate, hybridizing the plurality of samples of methylated DNA with labeled target binding probes specific for a target nucleic acid sequence and detecting the label of the labeled target binding probes and associating the label with the target nucleic acid sequence. Additionally, this invention provides a system for the detection of a target nucleic acid sequence including a flat substrate to bind methylated DNA in discrete patterns and a plurality of labeled target binding probes specific for a target nucleic acid sequence. The application for this technology, mirrors the application for PCR, which are well documented such as diagnostics, forensic, academic pursuits and so forth. However, because of the lack of dependence on enzamatic mechanisms, this method and system offers an alternative to areas where PCR has proven to be unreliable due to lack of extension, stringency concerns, etc.

### BRIEF DESCRIPTION OF THE DRAWINGS

A more complete understanding of the invention and its advantages will be apparent from the following Description of the Preferred Embodiment(s) taken in conjunction with the accompanying drawings, wherein:

FIG. 1 is an illustration of an automatic arrayer.

FIG. 2 is an illustration of a heating cassette.

FIG. 3 is a photo of a substrate with bound genomic DNA

FIG. 4 is photo of a portion of a substrate with bound genomic DNA.

### DETAILED DESCRIPTION OF THE PREFERRED EMBODIMENT

The present invention provides a method and system for printing, immobilizing, hybridizing, and detecting genomic DNA. All patents, patent applications and articles discussed or referred to in this specification are hereby incorporated by reference.

The following terms and acronyms are used throughout the detailed description:

1. Definitions

complementary—chemical affinity between nitrogenous bases as a result of hydrogen bonding. Responsible for the base pairing between nucleic acid strands. Klug, W. S. and Cummings, M. R. (1997) Concepts of Genetics, 5^{th} ed., Prentice-Hall, Upper Saddle River, N.J. (hereby incorporated by reference)

DNA (deoxyribonucleic acid)—The molecule that encodes genetic information. DNA is a double-stranded molecule held together by weak bonds between base pairs of nucleotides. The four nucleotides in DNA contain the bases: adenine (A), guanine (G) cytosine (C), and thymine (T). In nature, base pairs form only between A and T and between G and C; thus the base sequence of each single strand can be deduced from that of its partner.

genome—all the genetic material in the chromosomes of a particular organism; its size is generally given as its total number of base pairs.

genomic DNA—all of the genetic information encoded in a cell. Lehninger, A. L., Nelson, D. L. Cox, M. M. (1993) Principles of Biochemistry, 2^{nd} ed., Worth Publishers, New York, N.Y. (hereby incorporated by reference)

genotype—genetic constitution of an individual cell or organism.

heating cassette—housing mechanism for glass substrates while heating.

imaging cassette—housing mechanism for glass substrate while imaging.

microarray imager—is a reader used to detect samples bound or affixed to a flat substrate.

microarray technology—is a hybridization-based process that allows simultaneous quantitation of many nucleic acid species, has been described (M. Schena, D. Shalon, R. W. Davis, and P. O. Brown, “Quantititative Monitoring Of Gene Expression Patterns With A Complementary DNA Microarray,” Science, 270(5235), 467-70, 1995; J. DeRisi, L. Penland, P. O. Brown, M. L. Bittner, P. S. Meltzer, M. Ray, Y, Chen, Y. A. Su, and J. M. Trent, “Use Of A Cdna Microarray To Analyze Gene Expressions Patterns In Human Cancer,” Nature Genetics, 14(4), 457-60 (“DeRisi”), 1996; M. Schena, D. Shalon, R. Heller, A Chai, P. O. Brown, and R. W. Davis, “Parallel Human Genome Analysis: Microarray-Based Expression Monitoring Of 100 Genes,” Proc. Natl. Acad. Sci. USA., 93(20), 10614-9, 1996) hereby incorporated by reference. This technique combines robotic spotting of small amounts of individual, pure nucleic acids species on a glass surface, hybridization to this array with multiple fluorescently labeled nucleic acids, and detection and quantitation of the resulting fluor tagged hybrids with a scanning confocal microscope. This technology was developed for studying gene expression.

recombinant DNA—A combination of DNA molecules of different origin that are joined using recombinant DNA technologies.

substrate—Any three dimensional material to which sample or probe may be deposited that may have reactive groups to aid in attachment.

The present invention provides a system and method for applying, immobilizing, and hybridizing and detecting genomic DNA on a flat substrate. More specifically, the invention is a platform technology that allows for the detection of genetic sequences in the genomic DNA or subsets of genomic DNA. A subset of genomic DNA is any portion of the genome that has been isolated from the entire genome. Typically, subsets of genomic DNA have been made by sonication, chemical means (e.g., alkaline solutions) or enzamatic digestion of the genomic DNA. Subsets of genomic DNA range in size from virtually intact DNA 3.0×10⁹ base pairs to 25 base pairs. Detection of genomic DNA can be achieved by applying methylated DNA, such as the genomic DNA, on it to a microarray substrate. Detection of sequences from other types of nucleic acids, such as mitochondrial DNA, chloroplastic DNA and RNA/DNA hybrids is also achieved by applying these elements to the surface of a microarray substrate. A substrate can be, but is not limited to, a flat slide that includes functional or reactive groups to bind the genomic DNA.

The Substrate

A substrate is optically flat so that it can be scanned with a laser and it includes a sufficient number of functional or reactive groups to bind the genomic DNA to be screened. The substrates may be glass, plastic, membranes, or a combination of the elements. Typically the substrates have some surface chemistry attached. These surface chemistries include by not limited to amine, aldehydes, polylysine, carboxyl, silanated, silyated, nitrocellulose or epoxy groups. The reactive groups covalently or non-covalently attach the nucleic acid to the surface of the substrate. In the preferred embodiment, aldehyde function groups (5.0×10¹²), reactive groups per cm² are affixed to optically flat glass slide. The slide (SMA-1000) is purchased from TeleChem (Sunnyvale, Calif.). While the illustrated embodiment employs a 25 mm×76 mm glass slide, such microscopic slides may be larger, such as 6×2, 4×8, etc. The genomic DNA samples are applied to the flat substrate to form discrete patterns. Typically these patterns are generated by samples being places onto the substrate surface in columns and rows. The columns and rows forms grids which can be further divided into smaller segments know as subgrids. The genomic DNA samples may be applied to form linear or in staggered rows to allow greater array density on the substrate.

Some functional groups exhibit a better binding of DNA. These function groups include aldehyde, amine, carboxyl, polylysine, silanated, silyated, epoxy and nitrocellulose surface chemistries. More specifically, with respect to aldehyde substrates they contain aldehyde groups which are covalently attached to the substrate. Amines (NH₂) found on the on the adenine, cytosine and guamine residues of DNA react with the aldehyde groups forming covalent bonds. Attachment is stabilized by a dehydration reaction (drying in low humidity) which leads to Schiff base formation. Specific and covalent end attachment provides highly stable and accessible attachment of DNA.

More specifically, with respect to epoxy coupling chemistry DNA molecules contain primary amine groups on the adenine, cytosine and guamine residues are used to bind to the epoxy funtionalized substrate. The amine groups (NH₂) react with the carbon on the epoxide group, forming a covalent bond between the DNA and the substrate.

More specifically, with respect to amine substrates amine substrates contain amine groups (NH3⁺) attached covalently to the substrate. The amines carry a positive charge at neutral pH, allowing attachment of DNA through the formation of ionic bonds with the negatively charged phosphate backbone. Electrostatic attachment is supplemented by treatment with ultraviolet light or heat, which induces covalent attachment of the DNA to the surface. The combination of electrostatic binding and covalent attachment couples the DNA to the substrate is a highly stable manner.

Immobilizing

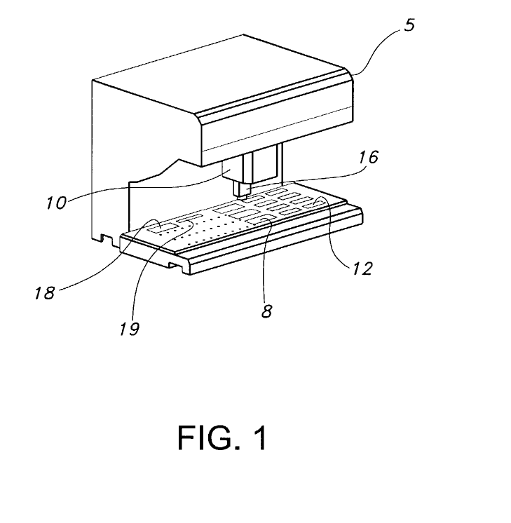

Genomic DNA once isolated is suspended in a water solution and salt. The genomic DNA solution is applied on the surface of the substrate with a solid pin tool using an automatic arrayer. An arrayer is a machine that dips stainless steel or titanium tips, or the like, into wells and prints on substrates. An automatic arrayer includes software that tracks the location of specific samples with its location on the substrate. An arrayer can be communicatively coupled to computer program such as Nautilis® (Thermal Lab System, Beverley, Mass.) which is a LIMS (Laboratory Information Management System) and information on each sample is transmitted to LIMS. Typically, automatic arrayers include, but are not limited, to solid pin, split pin/quill, tweezer, TeleChem's Micro Spotting Pin (Sunnyvale, Calif.), pin and ring, piezoelectric technology and syringe-solenoid technologies. An automatic arrayer can be used in this method according to the manufactures operating instructions without modification. Any arrayers can be used, such as Telechem's (Sunnyvale, Calif.) Spot Bot® to Genetix's (Queensway, United Kingdom) Qarray® machine, or the preferred embodiment of Dynamic Devices's (Newark, Deleware) Oasis machine. The Oasis microarrayer is shown in FIG. 1 as microarray 5. Microarray 5 includes a three axis (X, Y and Z) motion control instrument fitted with microfluidics delivery technology. The robotic delivery arm 10 can remove small amounts of sample from source plates 8 and deliver this sample to any number of substrates 12. The microarray 5 provides accurate and reproducible samples onto the substrate on the micron level. Also the microarray 5 has a computer tracking system that is flexible that allows for sample tracking. Additionally the microarrayer 5 is fitted with a cleaning station 14 that eliminates cross-contamination from one sample to the next.

The pin washing protocol on the microarray 5 involves several steps. The pins are suspended in the print head 16 which move about the deck of the machine. Washing begins with the pins being moved and submerged in distilled water in the sonicator 18. The sonicator 18 provides ultrasonic radiation. Sonication transpires for seven seconds in which the ultrasonic waves remove debris from the pin. The pins are then moved to a 70% ethanol bath for two seconds and then moves to the vacuum for 0.5 seconds. For the final wash the pins are submerged in the wash station 19 for 4 seconds and then vacuum dried for 12 seconds. The pins then pick up the next samples for printing.

With the aldehyde and Epoxy coated slides, the genomic DNA spots do not need to be processed further for attachment to the substrate after sufficient time or dehydration. However, using other functional groups, the genomic samples can be attached on the substrates by ultra-violetly cross-linked to the surface and/or thermally heating to attach the samples. For example, the genomic DNA is ultra-violetly attached to the substrate at 1200 μl for thirty seconds. Similarly, heating at 80° C. for 0.5-4 hours will also accomplish the attachment. The spots on the substrate are from between 1-10,000 microns in size. Between approximately 1-130,000 genomic DNA spots, corresponding to discrete trackable samples are located on an individual substrate. A discrete area is directly proportional to the printing technique used.

Genomic DNA contains reactive amines groups located on the adenine, cytosine and guamine bases in the DNA. Even though genomic DNA is methylated along some adenine and cytosine residues the genomic DNA is sufficiently localized, in the preferred embodiment, on the substrates contain primary aldehyde groups which are covalently attached to the glass surface. Amines (NH₂) found on the on the adenine, cytosine and guamine residues of DNA react with the aldehyde groups forming covalent bonds. Attachment is stabilized by a dehydration reaction (drying in low humidity) which leads to Schiff base formation. Specific and covalent end attachment provides highly stable and accessible attachment of DNA while maintaining its ability to hybridize with a probe.

Hybridizing

Once the genomic DNA is localized and sufficiently immobilized it undergoes a hybridization reaction. The genomic DNA is made single stranded either by chemical methodologies such as an alkaline solution or by heat. In the preferred embodiment the genomic DNA is denatured from its double stranded nature to a single stranded form by heating the DNA above 94° C. for 30 seconds to 30 minutes. The temperature above 90° C. breaks the two hydrogen bonds between the adenine and thymine and the three hydrogen bonds between guanine and cytosine. A labeled target binding probe, composed of nucleic acid, pairs complementarily to the nucleic acid segment of the single stranded genomic DNA of interest.

Detecting

A labeled target binding probe includes a label detectable by spectroscopic, photo chemical, biochemical, immunochemical or chemical means. Both direct labeling techniques and indirect labeling are contemplated. The label can be associated with the presence or amount of the target nucleic acid sequence.

Different techniques may be employed in order to label a probe. The indirect methodology as is described in U.S. Pat. Nos. 5,731,158; 5,583,001; 5,196,306 and 5,182,203 (hereby specifically incorporated by reference). In the direct labeling technique the labeled target probe hybridizes to the target nucleic acid sequence. The target binding probe will be directly modified to contain at least one fluorescent, radioactive or staining molecule per probe, such as cyanine, horseradish peroxidase (HRP) or any other fluorescent signal generation reagent. The fluorescent signal generation reagent includes, for example, FITC, DTAF and FAM. FAM is a fluorescein bioconjugate made of carboxyfluorescein succinimidyl ester (e.g. 5-FAM (Molecular Probes, Eugene, Oreg.). DTAF is a fluorescein dichlorotriazine bioconjugate.

The indirect labeling techniques uses a target binding probe that binds the selected nucleic acid target sequence and that has been modified to contain a specified epitope or if it has a nucleic acid binding sequence it forms a bipartite probe. In addition to the target sequence, an additional binding sequence beyond the specified target sequence is added. The combination of these two elements gives rise to a bipartite probe.

The preferred embodiment of the present invention involves genomic mouse DNA (3×10⁹ base pairs). The genomic mouse DNA can be isolated by both organic acid extraction (Phenol, chloroform, alcohol) and paramagnetic isolation with carboxylated Polysciences (Warrington, Pa.), Seradyn (Indianapolis, Ind.) and Agencourt (Beverly, Mass.) and silinated Promega (Madison, Wis.) beads. In the preferred embodiment, a paramagnetic isolation using a one micron carboxylated bead from Seradyn is employed. To the isolated DNA is added a printing buffer. The buffers includes 3× SSC, 5.5M Sodium Thiocynate (NaSCN), 1.7M Betaine, 50% Dimethyl Sulfoxide (DMSO), Sucrose, Foramide and 1× Telechem printing buffer. The preferred printing buffer is 3× SSC. The DNA with the printing buffer is printed on to Telechem's Superaldehyde substrates.

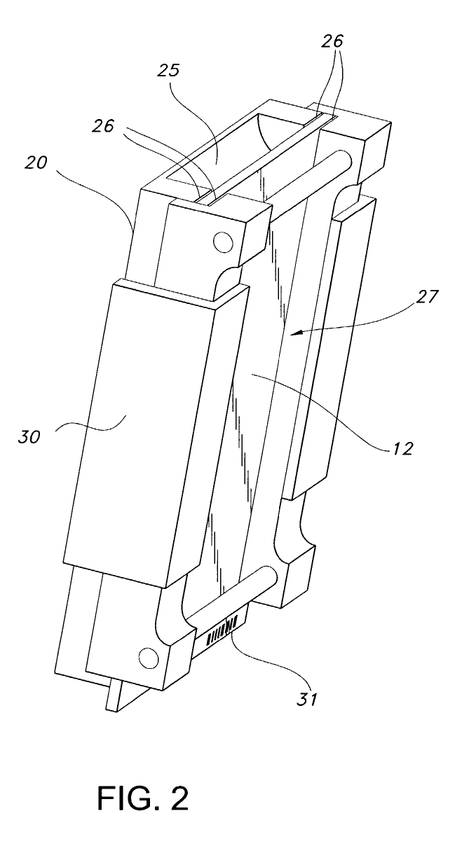

In the preferred embodiment, the printed substrate is loaded into a heating cassette as shown in FIG. 2. The heating cassette is composed of a beveled top plate, prefabricated spacers, a metal frame and tension clips. The substrate is lowered into the metal frame and spacers are placed on top of the substrate running lengthwise along the edge. The beveled top plate is then lowered on top of the substrate only separated by the spacers. The metal tension clips are then applied to the heating cassette, which holds the cassette together securely. The substrate 29 is placed in a heating cassette 20 for hybridization. Now referring to FIG. 2, a heating cassette 20 is shown, by way of example. This heating cassette 20 is made of a beveled top 25, a plurality of spacers 26, a metal frame 27 and tension clamps 30. The substrate 12 is lowered into the metal frame 27 and plastic spacers 26 are placed on top of the substrate 12 running lengthwise along the edge. The beveled top plate 25 is then lowered on around of the substrate 12 only separated by the plurality of spacers 26. The metal tension clamps 30 are then applied to the heating cassette 12, which hold the cassette 20 together securely. The barcode of the substrate 31 will extend beyond the heating cassette 20 to facilitate scanning.

The heating cassette 20 is assembled. The substrates 12 in the heating cassettes 20 are transferred to the heating block. The function of the heating block is to increase and decrease temperature. In the preferred embodiment, the heating block is heated to 95-99° C. for two minutes in order to separate the double stranded DNA making it more amenable to hybridization. The heating cassette 20 is placed on the exterior platform of the heating block. The heating block's exterior surface is thermally controlled by different temperature fluids being perfused by external circulator baths. The contact between the heating cassette substrate and the heating block permits a highly efficient thermal transfer. In the preferred embodiment, the heating block is heated to 95-120° C. for two minutes in order to separate the double stranded DNA making it more amenable to hybridization. In the preferred embodiment, the substrate 12 is then dried by forcing compressed filtered air into the top bevel of the heating cassette forcing out any residual fluid. A sufficient amount of Sodium Borohydrate, Casine, bovine serum albumine (BSA) or any commercial available blocking agent is dispensed to the bevel of the heating cassette 20 to block unbound surface chemistry, i.e. aldehydes. The heating cassette 20 is incubated on the heating block. Following the blocking of the surface chemistry with the blocking agent, the substrate is washed. In the preferred embodiment, the substrate is washed with de-ionized water for one minute three different times.

Blocking agents may or may not be added to the substrate to deactivate the unused surface chemistries before or after heating. Traditional blocking agents include, but not limited, Sodium Borohydrate (NaBH₄), Bovine Serum Albumin (BSA), Casine, or nucleic acids such as Herring sperm DNA, Cot1 DNA, single stranded DNA, Poly dA or Yeast tRNA. In the preferred embodiment, NaBH₄ is added to bevel of the heating cassette 20 and incubated for five minutes. Following the blocking of the surface chemistry with NaBH₄, the substrate is flushed with de-ionized water to remove the blocking agent. A hybridization solution is applied to the bevel top 25 of the heating cassette 20. A hybridization solution includes a labeled target binding probe specific for a target nucleic acid sequence in the sample of genomic DNA. A number of hybridization buffers are acceptable, such as water and saline sodium citrate (SSC). Alternatively, buffer solutions such as 0.25 NaPO₄, 4.5% SDS, 1mMEDTA, 1×SSC or 40% Formamide, 4×SSC, 1% SDS may also be used. The substrates 12 in the heating cassette 220 will be incubated. In the preferred embodiment, the hybridization mixture is incubated for between 0.5 to 12 hours at a temperature ranging from 40° C. to 65° C. on the heating block after the target binding probe. It should be noted that the hybridization solution can contain the amplification molecules or secondary signal reagents or they may be added secondarily.

Once the substrates 12 have been incubated with the hybridization solution the surface of the substrate is washed several times to remove any excess reagent such as probe amplification molecules or secondary signal reagents. In the preferred embodiment, the substrates 12 will first be washed and incubates at 55° C. with several volumes of 2× SSC, 0.2% SDS for ten minutes. The substrate will again be washed at room temperature for 10 minutes with several volumes of 2× SSC. The final wash will be conducted at room temperature for ten minutes with 0.2× SSC.

The substrate 12 is dried to facilitate imaging. In the preferred embodiment, the substrate is dried by forcing compress filtered air into the top bevel of the heating cassette, however centrifugation can be used. The compress filtered air drying will continue for several seconds until all of the residual buffer is forced out of the heating cassette and the substrate is dry.

The substrates 12 are loaded into a commercially available imaging cassette, such as GSI Lumonics (Watertown, Mass.) and the imaging cassettes are loaded into the microarray imager GSI Lumonics 5000 (Watertown, Mass.) used according to the manufacturer's instructions. In the preferred embodiment, Tecan's LS300 (Raleigh, N.C.) is used. The substrates 12 are exposed to an excitatory energy source to produce a quantifiable signal from the label. The quantifiable signal can be used to detect the presence or absence of the target nucleic acid sequence. Additionally, the amount of signal quantified can be correlated to the amount of nucleic acid target sequence present.

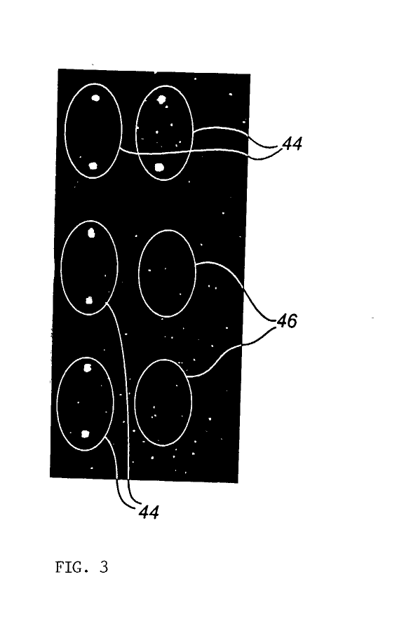

Now referring to FIG. 3, a photograph of a portion of a substrate 12 is shown. This substrate is glass functionalized with an aldehyde group made by Telechem (Sunnyvale, Calif.), brand name SuperAldehyde. The genomic DNA was isolated using Sambrook, J., Fritsch, E. F., and Maniatis, T., in Molecular Cloning: A Laboratory Manual. Cold Spring Harbor Laboratory Press, NY, Vol. 1, 2, 3 (1989 hereby specifically incorporated by reference). This image shows that genomic DNA can be immobilized in a discrete area as shown by a plurality of circles 46. Area 44 is approximately 4 pixels (340 microns) in size. Area 44 is spotted with mouse genomic DNA and is stained with a buffer including a dendrimer. The DNA was sonicated and stained with 3× SSC dendrimer. Sonication can be done by any conventional means such as a fixed horn instrument. Although there is a wide range of fragments from about 100 base pairs to up to 1 kilobase, the average size of the fragment is around about 500 base pairs (about meaning 50 base pairs).

### EXAMPLE 1

### DNA Purification

Three to nine milligrams of mouse biopsy was added to a 96 wellplate. To each well containing biopsy 180 μl of Promega's (Madison, Wis.) Nuclei Lysis Solution with three milligrams of Proteinase K per ml was added. The plate was move to a 55° C. oven and allowed to incubate for one hour. The plate was vortexed five seconds. 136 μl of lysate was removed from each well and placed into a clean 384 deep wellplate. 55 μl of mixed carboxylated Seradyn (Indianapolis, Ind.) particles supplied via Agencourt (Beverly, Mass.) was added to each well containing lysate. 187 μl of 20% polyethylene glycol (PEG) 8000, 0.02% sodium Azide and 2.5M Sodium Chloride was added to each sample. The samples were tip mixed three times with a volume of 250 μl. The samples were allowed to incubate at room temperature for ten minutes. The 384 deep wellplate was transferred to a magnetic surface for four minutes. The supernatant was removed leaving a pellet of particles at the bottom of each well. 200 μl of 70% ethanol wash solution was added to each well while still on the magnet. The particles were allowed to incubate three minutes. The 70% ethanol was removed and discarded. The wash process was repeated three more times. The particle pellets were allowed to dry in a 50° C. oven for 30 minutes. 30 μl of deionized water was added to each sample and allowed to incubate at room temperature for one minute. The samples were tip mixed eight times with a volume of 20 μl. The 384 deep wellplate was transferred back to the magnet for 1.5 minutes. 25 μl of eluate was transferred to a clean 96 UV optical wellplate. 5 ul of 20× Saline Sodium Citrate (SSC) was added to each sample in the optical plate. The samples were tip mixed three times with a volume of 25 μl. The optical plate was placed into an Optical Density reader (GENios; Serial number: 12900400173; Firmware: V 4.60-09/00 GENios; XFLUOR4 Version: V 4.20) and acquired 260 nm, 280 nm and 260/280 ratio reading).

TABLE 1

| <> | 1 | 2 | 3 | 4 | 5 | 6 | 7 | 8 | 9 | 10 |
|---|---|---|---|---|---|---|---|---|---|---|
| A | 0.4679 | — | — | — | — | — | — | — | — | — |
| B | 0.6729 | — | — | — | — | — | — | — | — | — |
| C | 0.4774 | — | — | — | — | — | — | — | — | — |
| D | 0.7939 | — | — | — | — | — | — | — | — | — |
| E | 0.3583 | — | — | — | — | — | — | — | — | — |
| F | 0.9081 | — | — | — | — | — | — | — | — | — |
| G | 0.4244 | — | — | — | — | — | — | — | — | — |
| H | 0.4975 | — | — | — | — | — | — | — | — | — |
| I | 0.5794 | — | — | — | — | — | — | — | — | — |
| J | 0.7966 | — | — | — | — | — | — | — | — | — |
| K | 0.4910 | — | — | — | — | — | — | — | — | — |
| L | 0.6325 | — | — | — | — | — | — | — | — | — |
| M | | — | — | — | — | — | — | — | — | — |
| N | | — | — | — | — | — | — | — | — | — |
| O | | — | — | — | — | — | — | — | — | — |
| P | | — | — | — | — | — | — | — | — | — |

Dual wave data (difference)

TABLE 2

| | | 2 | 3 | 4 | 5 | 6 | 7 | 8 | 9 | 10 |
|---|---|---|---|---|---|---|---|---|---|---|
| A | 0.6044 | — | — | — | — | — | — | — | — | — |
| B | 1.5218 | — | — | — | — | — | — | — | — | — |
| C | 0.6102 | — | — | — | — | — | — | — | — | — |
| D | 1.4098 | — | — | — | — | — | — | — | — | — |
| E | 0.4841 | — | — | — | — | — | — | — | — | — |
| F | 1.8873 | — | — | — | — | — | — | — | — | — |
| G | 0.6301 | — | — | — | — | — | — | — | — | — |
| H | 0.7039 | — | — | — | — | — | — | — | — | — |
| I | 0.6960 | — | — | — | — | — | — | — | — | — |
| J | 1.1770 | — | — | — | — | — | — | — | — | — |
| K | 0.6078 | — | — | — | — | — | — | — | — | — |
| L | 1.3739 | — | — | — | — | — | — | — | — | — |
| M | | — | — | — | — | — | — | — | — | — |
| N | | — | — | — | — | — | — | — | — | — |
| O | | — | — | — | — | — | — | — | — | — |
| P | | — | — | — | — | — | — | — | — | — |

Raw data (dual wave measurement with measurement filter)

TABLE 3

| | | 2 | 3 | 4 | 5 | 6 | 7 | 8 | 9 | 10 |
|---|---|---|---|---|---|---|---|---|---|---|
| A | 0.1365 | — | — | — | — | — | — | — | — | — |
| B | 0.8489 | — | — | — | — | — | — | — | — | — |
| C | 0.1328 | — | — | — | — | — | — | — | — | — |
| D | 0.6159 | — | — | — | — | — | — | — | — | — |
| E | 0.1258 | — | — | — | — | — | — | — | — | — |
| F | 0.9792 | — | — | — | — | — | — | — | — | — |
| G | 0.2057 | — | — | — | — | — | — | — | — | — |
| H | 0.2064 | — | — | — | — | — | — | — | — | — |
| I | 0.1166 | — | — | — | — | — | — | — | — | — |
| J | 0.3804 | — | — | — | — | — | — | — | — | — |
| K | 0.1168 | — | — | — | — | — | — | — | — | — |
| L | 0.7414 | — | — | — | — | — | — | — | — | — |
| M | | — | — | — | — | — | — | — | — | — |
| N | | — | — | — | — | — | — | — | — | — |
| O | | — | — | — | — | — | — | — | — | — |
| P | | — | — | — | — | — | — | — | — | — |

Raw data (dual wave measurement with reference filter)

TABLE 4

| <> | 1 | 2 | 3 | 4 | 5 | 6 | 7 | 8 | 9 | 10 |
|---|---|---|---|---|---|---|---|---|---|---|
| A | 0.2487 | — | — | — | — | — | — | — | — | — |
| B | 0.3632 | — | — | — | — | — | — | — | — | — |
| C | 0.2522 | — | — | — | — | — | — | — | — | — |
| D | 0.3982 | — | — | — | — | — | — | — | — | — |
| E | 0.1880 | — | — | — | — | — | — | — | — | — |
| F | 0.4814 | — | — | — | — | — | — | — | — | — |
| G | 0.2211 | — | — | — | — | — | — | — | — | — |
| H | 0.2999 | — | — | — | — | — | — | — | — | — |
| I | 0.3024 | — | — | — | — | — | — | — | — | — |
| J | 0.4799 | — | — | — | — | — | — | — | — | — |
| K | 0.2581 | — | — | — | — | — | — | — | — | — |
| L | 0.3920 | — | — | — | — | — | — | — | — | — |
| M | | — | — | — | — | — | — | — | — | — |
| N | | — | — | — | — | — | — | — | — | — |
| O | | — | — | — | — | — | — | — | — | — |
| P | | — | — | — | — | — | — | — | — | — |

Dual wave data (difference)

TABLE 5

| <> | 1 | 2 | 3 | 4 | 5 | 6 | 7 | 8 | 9 | 10 |
|---|---|---|---|---|---|---|---|---|---|---|
| A | 0.3855 | — | — | — | — | — | — | — | — | — |
| B | 0.6432 | — | — | — | — | — | — | — | — | — |
| C | 0.3860 | — | — | — | — | — | — | — | — | — |
| D | 0.9917 | — | — | — | — | — | — | — | — | — |
| E | 0.3143 | — | — | — | — | — | — | — | — | — |
| F | 1.4484 | — | — | — | — | — | — | — | — | — |
| G | 0.4238 | — | — | — | — | — | — | — | — | — |
| H | 0.4689 | — | — | — | — | — | — | — | — | — |
| I | 0.4187 | — | — | — | — | — | — | — | — | — |
| J | 0.8534 | — | — | — | — | — | — | — | — | — |
| K | 0.3765 | — | — | — | — | — | — | — | — | — |
| L | 1.0643 | — | — | — | — | — | — | — | — | — |
| M | | — | — | — | — | — | — | — | — | — |
| N | | — | — | — | — | — | — | — | — | — |
| O | | — | — | — | — | — | — | — | — | — |
| P | | — | — | — | — | — | — | — | — | — |

Raw data (dual wave measurement with measurement filter)

TABLE 6

| | | | | | | | | | | 10 |
|---|---|---|---|---|---|---|---|---|---|---|
| A | 0.1368 | — | — | — | — | — | — | — | — | — |
| B | 0.2800 | — | — | — | — | — | — | — | — | — |
| C | 0.1338 | — | — | — | — | — | — | — | — | — |
| D | 0.5935 | — | — | — | — | — | — | — | — | — |
| E | 0.1263 | — | — | — | — | — | — | — | — | — |
| F | 0.9670 | — | — | — | — | — | — | — | — | — |
| G | 0.2027 | — | — | — | — | — | — | — | — | — |
| H | 0.1690 | — | — | — | — | — | — | — | — | — |
| I | 0.1163 | — | — | — | — | — | — | — | — | — |
| J | 0.3735 | — | — | — | — | — | — | — | — | — |
| K | 0.1184 | — | — | — | — | — | — | — | — | — |
| L | 0.6723 | — | — | — | — | — | — | — | — | — |
| M | | — | — | — | — | — | — | — | — | — |
| N | | — | — | — | — | — | — | — | — | — |
| O | | — | — | — | — | — | — | — | — | — |
| P | | — | — | — | — | — | — | — | — | — |

Raw data (dual wave measurement with reference filter)

The most commonly used methods of determining nucleic acid concentration is by performing an absorbance reading at 260 nm. Proteins have a tendency to absorb light at 280 nm. Table 2 represents the raw data reading for 260 nm and Table 5 represents the raw data reading for 280 nm. Since all substances, such as water and the optical plate, have some degree of a natural ability to absorb light, a reference wavelength should be used. Table 3 and Table 6 represents the data associated with a 999 nm reference wavelength reading. These values indicate the naturally occurring background noise. Table 1 (260 nm) represents the difference between Table 2 and Table 3. Table 4 (280 nm) represents the difference between Table 5 and Table 6. Subtracting the background noise from the raw yields a more accurate reading for both 260 nm and 280 nm. Table 7 represents the 260 nm/280 nm ratio. Nucleic acids absorb light at 260 nm and proteins absorb at 280 nm resulting in values that indicate the quantity of each substance. Dividing the DNA yield by the protein yield gives the DNA quality in terms of protein contamination. Stringent chemistries such a PCR and Sequencing are very intolerant of protein contamination. Typical acceptable ratio values for these reactions is 1.8 or greater.

TABLE 7

| 260 | 280 | ratio |
|---|---|---|
| 0.4679 | 0.2487 | 1.881383 |
| 0.6729 | 0.3632 | 1.852698 |
| 0.4774 | 0.2522 | 1.892942 |
| 0.7939 | 0.3982 | 1.993722 |
| 0.3583 | 0.1880 | 1.905851 |
| 0.9081 | 0.4814 | 1.886373 |
| 0.4244 | 0.2211 | 1.919493 |
| 0.4975 | 0.2999 | 1.658886 |
| 0.5794 | 0.3024 | 1.916005 |
| 0.7966 | 0.4799 | 1.659929 |
| 0.4910 | 0.2581 | 1.902363 |
| 0.6325 | 0.3920 | 1.61352 |

The samples were transferred into a 384 polypropylene V-bottom plate and loaded onto the microarrayer. Four of Telechem's (Sunnyvale, Calif.) Stealth 10B pins were used to print the mouse genomic DNA onto a slide. Five replicates of each sample were printed 750 um apart. After printing onto Superaldehyde (Telechem, Sunnyvale, Calif.) slides the samples were transfer to a desicator at 30% humidity for 60 minutes.

### EXAMPLE 2

### Hybridization

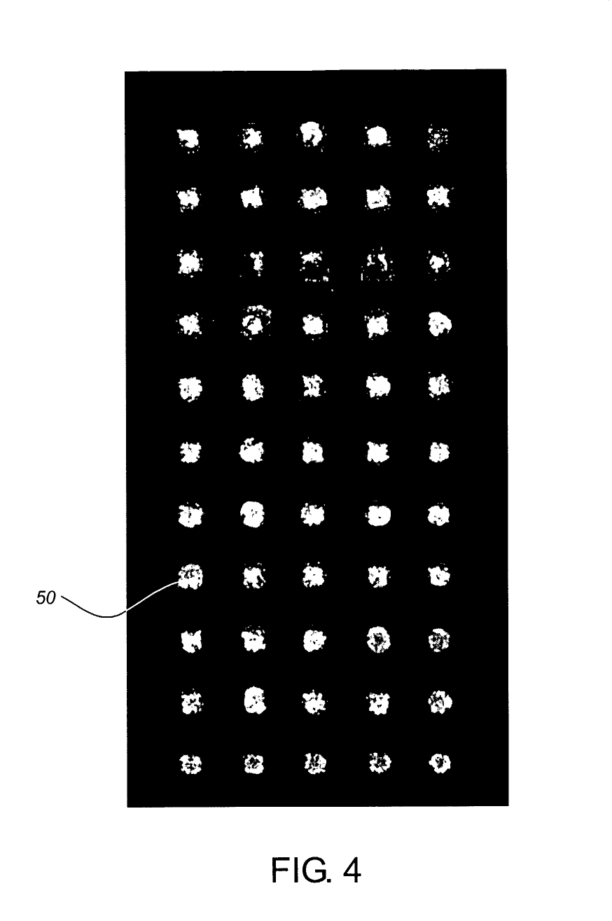

In another example as shown in FIG. 4, mouse genomic DNA is immobilized onto the surface of a Superaldehyde substrate. The Mouse genomic DNA was mixed with 20× SSC to produce an overall solution of 3× SSC. The DNA was printed onto Telechem's Superaldehyde substrate with a SpotBot and Stealth 10 Pins, also from Telechem. The printed DNA was allowed to dry in a desicator for 60 minutes in 30% humidity. The slide was then washed four minutes in deionized water. The slide was then boiled for five minutes in deionized water. The remaining reactive groups were removed from the slide by immersing the slide in Sodium Borohydrate (1.0 g NaBH₄, 88 mls 100% ethanol, 300 mls of PBS) for five minutes followed by a one minute wash in deionized water. 0.9 μl of 200 μM bipartite, specific for a housekeeping gene, was added to 29.1 μl of 0.25 NaPO₄, 4.5% SDS, 1mM EDTA, 1× SSC, coverslipped and incubated at 52° C. for 60 minutes to provide a labeled target binding probe specific for a control sequence of DNA. The substrate was then removed and washed with 2× SSC for 3 minutes at room temperature followed by a 0.2× SSC wash for 1 minute at room temperature. 2.5 μl of CY3 dendrimer and 27.5 μl of 40% Formamide, 4×SSC, 1% SDS, 2× Denhardt's Solution, coverslipped and incubated at 52° C. for 60 minutes. The excess dendrimer was removed with washes of 2× SSC, 0.2% SDS for 15 minutes at room temperature, 2× SSC washes for 15 minutes at room temperature and a 0.2× SSC wash for 1 min at room temperature. The substrate was dried and scanned. The image shows the detection of an endogenous gene in the mouse genome which has been localized into a discrete area 50.

Although the present invention has been described and illustrated with respect to preferred embodiments and a preferred use thereof, it is not to be so limited since modifications and changes can be made therein which are within the full scope of the invention.

We claim:

1. A method for detecting a target nucleic acid sequence comprising:
(a) applying a plurality of samples of methylated DNA on to a flat substrate to form discrete patterns of said methylated DNA onto said flat substrate;
(b) hybridizing said plurality samples of methylated DNA with labeled target binding probes specific for a target nucleic acid sequence;
(c) detecting the label of said labeled target binding probes; and
(d) associating said label with said target nucleic acid sequence.

2. The method of claim 1 wherein said discrete patterns are 50 to 10,000 microns in diameter.

3. The method of claim 1 wherein said plurality of samples of methylated DNA is immobilized on said substrate.

4. The method of claim 1 wherein said substrate is functionalized with a chemical selected from the group consisting of: aldehyde, amine, carboxyl, polylysine, silanated, silyated, epoxy and nitrocellulose.

5. The method of claim 1 wherein said methylated DNA is genomic DNA.

6. The method of claim 1 wherein said methylated DNA is a subset of genomic DNA.

7. The method of claim 1 wherein said methylated DNA is sonicated to form subsets of genomic DNA.

8. The method of claim 1 wherein the said flat substrate is a glass slide.

9. A system for the detection of a target nucleic acid sequence comprising:
(a) a flat substrate to bind methylated DNA in discrete patterns; and
(b) a plurality of labeled target binding probes specific for a target nucleic acid sequence.

10. The system of claim 9 wherein said discrete patterns are 50 to 10,000 microns in diameter.

11. The system of claim 9 wherein said substrate is functionalized with a chemical selected from the group consisting of: aldehyde, amine, carboxyl, polylysine, silanated, silyated, epoxy and nitrocellulose.

12. The system of claim 9 wherein said methylated DNA is genomic DNA.

13. The system of claim 9 wherein said methylated DNA is a subset of genomic DNA.

14. The system of claim 9 wherein said methylated DNA is sonicated to form subsets of genomic DNA.

15. The system of claim 9 wherein the said flat substrate is a glass slide.

16. The method of claim 9 wherein said system further includes a plurality of labeled target binding probes specific for a control sequence of DNA.

17. The method of claim 1 wherein said methylated DNA is selected from the group consisting of: mitochondrial DNA, chloroplastic DNA and DNA/RNA hybrids.
